(19) United States
(12) Patent Application Publication
(10) Pub. No.: US 2019/0125163 A1
(43) Pub. Date: May 2, 2019

(54) METHOD AND APPARATUS FOR ANALYZING IMAGES OF CAPSULE ENDOSCOPE BASED ON KNOWLEDGE MODEL OF GASTROINTESTINAL TRACT DISEASES

(71) Applicant: AJOU UNIVERSITY INDUSTRY-ACADEMIC COOPERATION FOUNDATION, Suwon-si (KR)

(72) Inventors: Jung Won LEE, Seoul (KR); Ye Seul PARK, Incheon (KR); Gyu Bon HWANG, Yongin-si (KR)

(73) Assignee: AJOU UNIVERSITY INDUSTRY-ACADEMIC COOPERATION FOUNDATION, Suwon-si (KR)

(21) Appl. No.: 16/170,257

(22) Filed: Oct. 25, 2018

(30) Foreign Application Priority Data
Oct. 26, 2017 (KR) 10-2017-0140438

(51) Int. Cl. A61B 1/00; G06T 7/00; A61B 1/04

(52) U.S. Cl. CPC A61B 1/00009; G06T 7/0012; A61B 1/041; G06T 2207/20104; G06T 2207/10068; G06T 2207/30092; A61B 1/00045

(57) ABSTRACT

According to an exemplary embodiment of the present disclosure, a capsule endoscopic image analyzing method includes: receiving signs from a user, by a capsule endoscopic image analyzing apparatus; determining a disease which shows the signs using disease information included in a knowledge model, by the capsule endoscopic image analyzing apparatus; determining findings which are found from a gastrointestinal tract due to the disease using findings included in the knowledge model, by the capsule endoscopic image analyzing apparatus; and separately providing only frames in which the findings appear in an image photographed by a capsule endoscope, by the capsule endoscopic image analyzing apparatus.

### CROSS-REFERENCE TO RELATED APPLICATIONS

This application claims the priority of Korean Patent Application No. 10-2017-0140438 filed on Oct. 26, 2017, in the Korean Intellectual Property Office, the disclosure of which is incorporated herein by reference.

### BACKGROUND

### Field

The present disclosure relates to a method and an apparatus for analyzing images of a capsule endoscope based on a knowledge model and more particularly, a method for providing clinical information by analyzing images of a capsule endoscope based a knowledge model defined on the basis of a minimal standard terminology for gastrointestinal endoscopy (MST) and a capsule endoscopy structured terminology (CEST) and an apparatus for performing the same.

### Description of the Related Art

A capsule endoscope is a device which is inserted into a human body to observe a gastrointestinal tract and photograph an image in the body. Therefore, it is necessary to identify a position (esophagus, stomach, jejunum, and ileum) of the capsule and information (tumors or polyps) found from the corresponding position to analyze the images of the capsule endoscope. However, due to the nature of the digestive system, when a capsule endoscopy of one patient is performed, images for 14 to 18 hours are obtained so that it takes so much time to analyze a vast amount of images. Therefore, a diagnostic radiologist reads images to be analyzed while narrowing the range of the images to be analyzed by focusing on a suspected disease of the patient, thereby analyzing the capsule endoscopic images.

The suspected diseases of the patient are inferred diseases by ontology for major lesions which should be found from the capsule endoscopic images and may be reasoned from signs prior to performing the capsule endoscopy. When such a suspected disease (or estimated disease) is selected, endoscopic characteristics which may be found from the suspected disease may be identified. For example, according to a typical opinion of Crohn's disease, a discrete ulcer with a relative distinct border is observed between normal mucosa in a longitudinal orientation so that the images may be analyzed by pay attention to the fact that such lesions are intermittently found.

However, the currently used capsule endoscopy software provides simply just image information without considering the relation between the suspected disease and major lesions found from the images. Therefore, needs for methods for providing clinical information during an image analyzing process of a capsule endoscope based on a knowledge model are increasing.

### SUMMARY

A technical object to be achieved by the present disclosure is to provide a method and an apparatus for analyzing images of a capsule endoscope based on a knowledge model.

Technical objects of the present invention are not limited to the aforementioned technical objects and other technical objects which are not mentioned will be apparently appreciated by those skilled in the art from the following description.

According to an aspect of the present disclosure, a capsule endoscopic image analyzing method includes: receiving signs from a user, by a capsule endoscopic image analyzing apparatus; determining a disease which shows the signs using disease information included in a knowledge model, by the capsule endoscopic image analyzing apparatus; determining findings which are found from a gastrointestinal tract due to the disease using finding information included in the knowledge model, by the capsule endoscopic image analyzing apparatus; and separately providing only frames in which the findings appear in images photographed by a capsule endoscope, by the capsule endoscopic image analyzing apparatus.

Desirably, the capsule endoscopic image analyzing method may further include defining a relation between signs and a disease and a relation between the disease and findings in advance, based on the ontology, by the capsule endoscopic image analyzing apparatus.

Desirably, the separately providing of only frames may include preparing a report, defined in capsule endoscopy structured terminology (CEST), including the frame as an image form and information on the disease and the findings as a text form.

Desirably, a plurality of findings is provided and the report further includes statistic information on the number and the size for the plurality of findings.

Desirably, a plurality of diseases is provided and the report further includes statistic information of an actual occurring probability of each disease according to a matching rate of each disease included in the plurality of diseases and the findings.

Desirably, the receiving of signs from a user may include additionally receiving symptoms from a medical information system.

Desirably, the separately providing of only frames may include visually providing a first region which provides the overall image photographed by the capsule endoscope and a second region which separately displays only the frame around the first region through a graphic user interface (GUI).

According to another aspect of the present disclosure, a capsule endoscopic image analyzing apparatus includes an input unit which receives signs from a user; a sign analyzing unit which determines a disease showing the signs using disease information included in a knowledge model; an image analyzing unit which determines findings which are found from a gastrointestinal tract due to the disease using finding information included in the knowledge model; and a disease information output unit which separately provides only frames in which the findings appear in an image photographed by a capsule endoscope.

Effects according to the present disclosure are as follows:

The present disclosure proposes a gastrointestinal tract disease model and an analyzing system which enable intelligent capsule endoscopic software based on knowledge information on a suspected disease. According to the present disclosure, when a specific patient shows signs of elevation of leukocytes, anemia, diarrhea, fever, and C-reactive protein, Crohn's disease may be determined as the suspected disease of the patient based on the gastrointestinal tract disease model. In this case, in order to analyze the images for Crohn's disease, characteristics for a longitudinal ulcer, a cobblestone appearance, or an inflammatory polyposis are presented as related finding information to assist the diagnosis of the doctor. By doing this, the convenience of medical staffs who use the capsule endoscope, such as image analysts, diagnostic radiologists, and doctors, may be improved not only by showing images photographed by the capsule endoscope through a viewer program, but also by providing clinical information together with the images.

The effects of the present disclosure are not limited to the technical effects mentioned above, and other effects which are not mentioned can be clearly understood by those skilled in the art from the following description

### BRIEF DESCRIPTION OF THE DRAWINGS

The above and other aspects, features and other advantages of the present disclosure will be more clearly understood from the following detailed description taken in conjunction with the accompanying drawings, in which:

FIGS. 1 to 3 are views for explaining an image analyzing method of a capsule endoscope based on a disease model according to an exemplary embodiment of the present disclosure;

FIGS. 4 to 7 are views for explaining a disease model available for an exemplary embodiment of the present disclosure in more detail;

FIG. 8 is a view for explaining an image analyzing apparatus of a capsule endoscope based on a disease model according to an exemplary embodiment of the present disclosure;

FIGS. 9A to 9G are views for explaining a knowledge model used in an exemplary embodiment of the present disclosure; and

FIG. 10 is a flowchart for explaining an image analyzing method of a capsule endoscope based on a disease model according to an exemplary embodiment of the present disclosure.

### DETAILED DESCRIPTION OF THE PREFERRED EMBODIMENT

Those skilled in the art may make various modifications to the present invention and the present invention may have various embodiments thereof, and thus specific embodiments will be illustrated in the drawings and described in detail in the detailed description. However, this does not limit the present invention within specific exemplary embodiments, and it should be understood that the present invention covers all the modifications, equivalents and replacements within the spirit and technical scope of the present invention. In the description of respective drawings, similar reference numerals designate similar elements.

Terms such as first, second, A, or B may be used to describe various components but the components are not limited by the above terms. The above terms are used only to discriminate one component from the other component. For example, without departing from the scope of the present invention, a first component may be referred to as a second component, and similarly, a second component may be referred to as a first component. A term of and/or includes a combination of a plurality of related elements or any one of the plurality of related elements.

It should be understood that, when it is described that an element is “coupled” or “connected” to another element, the element may be directly coupled or directly connected to the other element or coupled or connected to the other element through a third element. In contrast, when it is described that an element is “directly coupled” or “directly connected” to another element, it should be understood that no element is present therebetween.

Terms used in the present application are used only to describe a specific exemplary embodiment, but are not intended to limit the present invention. A singular form may include a plural form if there is no clearly opposite meaning in the context. In the present invention, it should be understood that terminology “include” or “have” indicates that a feature, a number, a step, an operation, a component, a part or the combination those of described in the specification is present, but does not exclude a possibility of presence or addition of one or more other features, numbers, steps, operations, components, parts or combinations, in advance.

If it is not contrarily defined, all terms used herein including technological or scientific terms have the same meaning as those generally understood by a person with ordinary skill in the art. Terms defined in generally used dictionary shall be construed that they have meanings matching those in the context of a related art, and shall not be construed in ideal or excessively formal meanings unless they are clearly defined in the present application.

Hereinafter, exemplary embodiments according to the present invention will be described in detail with reference to accompanying drawings.

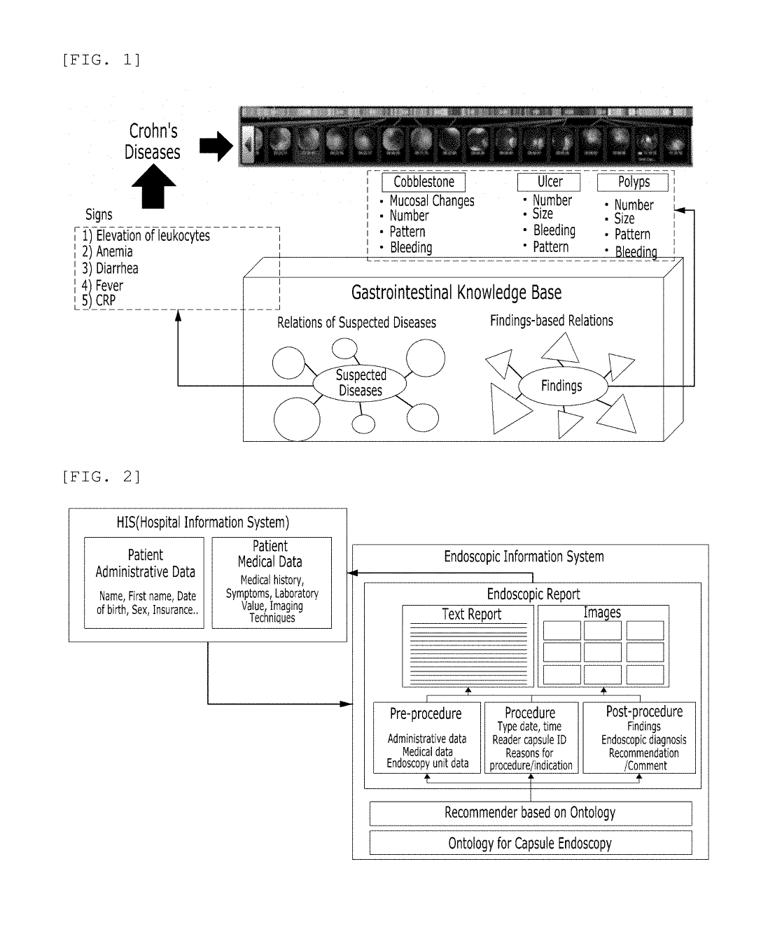

FIGS. 1 to 3 are views for explaining an image analyzing method of a capsule endoscope based on a disease model according to an exemplary embodiment of the present disclosure.

Referring to FIG. 1, a disease model used in the present disclosure is illustrated in the lower portion. The disease model is also called a knowledge model. Referring to FIG. 1, the disease model used in the present disclosure is configured by suspected diseases and endoscopic findings. The suspected diseases may be reasoned through one or more signs. The endoscopic findings may be reasoned through the suspected diseases. That is, the subsequent reasoning is performed through the steps of signs>suspected diseases>endoscopic findings.

For example, patients using a capsule endoscope may mention signs that they are experiencing through a medical interview before inserting the capsule endoscope into an oral cavity. In an example of FIG. 1, when a patient shows signs of elevation of leukocytes, anemia, diarrhea, fever, and C-reactive protein, Crohn's disease may be reasoned as the suspected disease of the patient. Next, findings mainly observed from endoscopic images of patients with Crohn's diseases may be reasoned through a previously learned model. For example, a longitudinal ulcer, a cobblestone appearance, and an inflammatory polyposis are reasoned and frames where the corresponding findings are photographed in the capsule endoscopic images of the patient may be mainly provided to the medical staffs. By doing this, an indicator for determining whether the Crohn's disease which is a suspected disease actually occurs may be provided.

Referring to the upper portion of FIG. 1, frames of capsule endoscopic images over 14 hours are illustrated and only specific sections are enlarged in the lower portion by selecting only frames from which findings related to the Crohn's disease which is the suspected disease are found, among them. Further, statistic information on the sections where the findings are found may be generated and provided. For example, information such as the number of found longitudinal ulcers, a size, bleeding, and a pattern may be additionally provided together with image information.

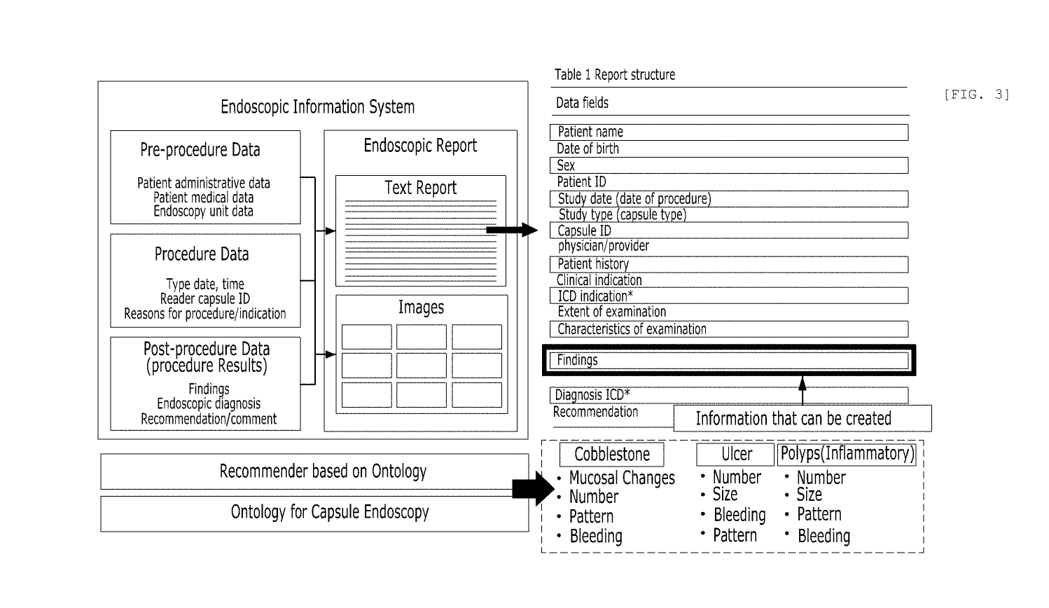

FIG. 2 is a view of a system implementing an image analyzing method of a capsule endoscope based on a disease model proposed by the present disclosure. Referring to FIG. 2, an endoscopic information system interworks with a hospital information system. The endoscopic information system proposed by the present disclosure interworks with the hospital information system to exchange medical information therebetween. Data received from the hospital information system includes administrative data (for example, name, age, and sex) for identifying a patient and medical data (for example, a medical history, symptoms, and test results). Further, data transmitted from the endoscopic information system is information on an endoscopic report including information on a pre-procedure, a procedure, and a post-procedure.

In this case, the endoscopic information system may manage a knowledge model based on MST and CEST. The minimal standard terminology for gastrointestinal endoscopy abbreviated as MST is a minimal standard terminology definition for gastrointestinal endoscopy established by World Endoscopy Organization and is a terminology standard for an endoscopic information system. Currently, it is recommended to develop the endoscopic information system utilizing the standard terminologies and the contents define not only information on lesions which may be found from the endoscopy, but also terminologies of the reasons for endoscopy, post-treatments, and events.

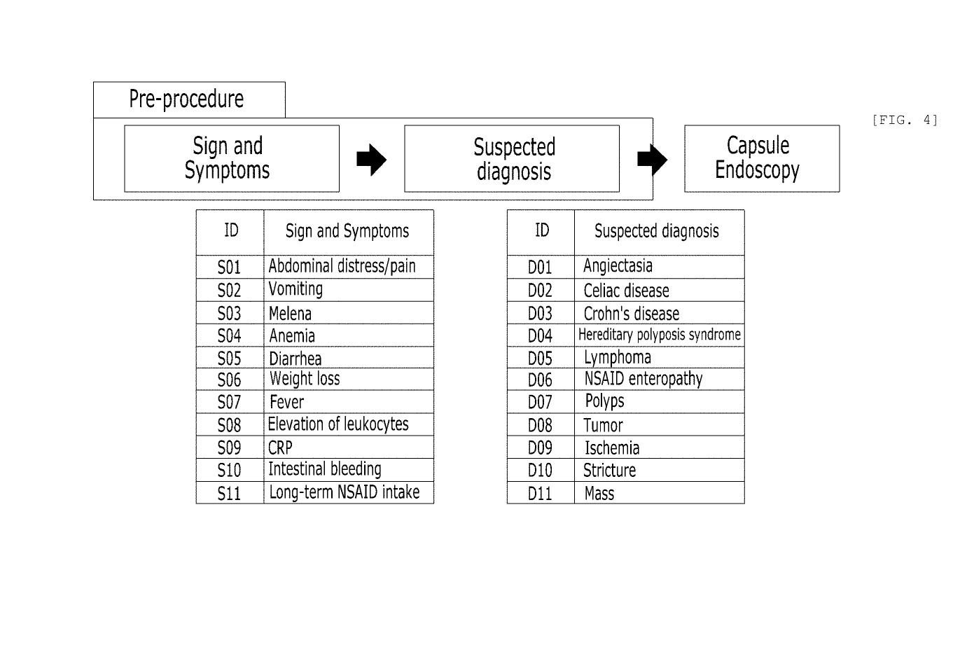

Capsule endoscopy structured terminology abbreviated as CEST is a terminology standard which is focusing only on the capsule endoscopy, rather than the entire endoscopy and the proposed standard suggests a report standard for preparing the reading results of capsule endoscopic images. A detailed structure of the report to be prepared is as illustrated in FIG. 3, which means a structure for a text report of the endoscopic information system illustrated in FIG. 2.

Referring to FIG. 2 again, the endoscopic information system interworks with the hospital information system (HIS) to receive patient administrative data and patient medical data and recommend clinical information of the patient by applying a knowledge model based on ontology thereto. For example, a suspected disease is proposed based on the patient's sign information or if there is a suspected disease, predicted finding information may be proposed. The medical staffs may provide an actual medical service based on the proposed information. Specifically, MST or CEST of the related art is established for standards and concerns only definitions of terminologies so that there is a lack of interest in the relation therebetween. In order to improve the above disadvantage, according to the present disclosure, the relation between lesions or the relation with causes (diseases or prognostic factors) is defined by a knowledge model based on the ontology and when a particular item of MST or CEST is found therethrough, data of another item related to the found particular item is automatically reasoned and proposed. Specifically, since the ontology utilized during this process is defined based on the terminology proposed in MST and CEST standard, the clinical information to be reasoned or proposed is also a standard terminology.

Referring to FIG. 3, it is understood that the endoscopic report illustrated on the right side of the endoscopic information system of FIG. 2 is more specifically illustrated. The endoscopic report is created to include a text report and images for items illustrated in Table 1 of FIG. 3, based on MST and CEST. In this case, it is necessary to note the detected findings represented in Table 1. Findings such as ulcers, hemorrhages, mucosal lesions, and tumors found from the capsule endoscopic images are necessary for a report. The image analyzing method of a capsule endoscope based on a disease model proposed in the present disclosure automatically searches findings from the images based on the knowledge model and provides clinical information for preparing a report.

That is, when the method proposed by the present disclosure is used, a screening process for checking whether findings related to the suspected disease are actually found from the images photographed by the capsule endoscope is automatically performed to assist the reading of the capsule endoscopic images and the results are collected to provide the clinical information. By doing this, the convenience of examiners may be improved.

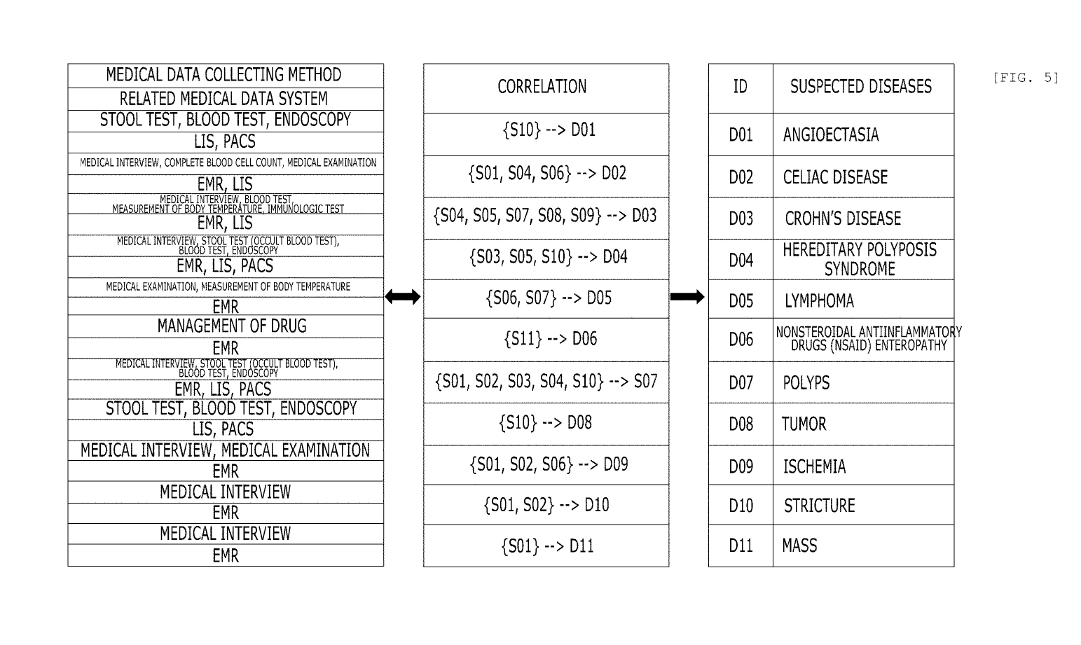

FIGS. 4 to 7 are views for explaining a disease model available for an exemplary embodiment of the present disclosure in more detail.

Referring to FIG. 4, as a pre-procedure before inserting the capsule endoscope into an oral cavity of the patient, signs and symptoms are input and suspected diseases are selected based on the signs and symptoms, from the knowledge model. To this end, signs and symptoms and suspected diseases need to be built in advance by the ontology.

Referring to Table on a left side of FIG. 4, various signs and symptoms S01 to S11 are enumerated. For example, signs such as abdominal distress/pain, vomiting, melena, anemia, diarrhea, weight loss, fever, elevation of leukocytes, C-reactive protein, intestinal bleeding, long-term NSAID intake are enumerated. Among them, the patient may input signs of S01 to S05 to a system through a medical interview before performing an examination using a capsule endoscope. Further, a medical staff who is in charge of the patient inputs symptoms of S6 to S11.

Various suspected diseases such as D01 to D11 illustrated in Table on a right side may be selected based on the signs and symptoms input as described above. For example, suspected diseases such as angiectasia, celiac disease, Crohn's disease, hereditary polyposis syndrome, lymphoma, NSAID enteropathy, polyps, tumor, ischemia, stricture, or mass may be selected. In this case, a plurality of suspected diseases related to the signs and symptoms may be selected, because some diseases show similar signs or symptoms.

In this case, when a report which analyzes the capsule endoscopic images is provided to the medical staff, statistic information indicating an actual disease having a high probability through a ratio of each disease and the findings found in the actual capsule endoscopic image may be created to be provided. The medical staff may diagnose the actual disease by reflecting a medical opinion to the statistic information and provide medical information to the patient.

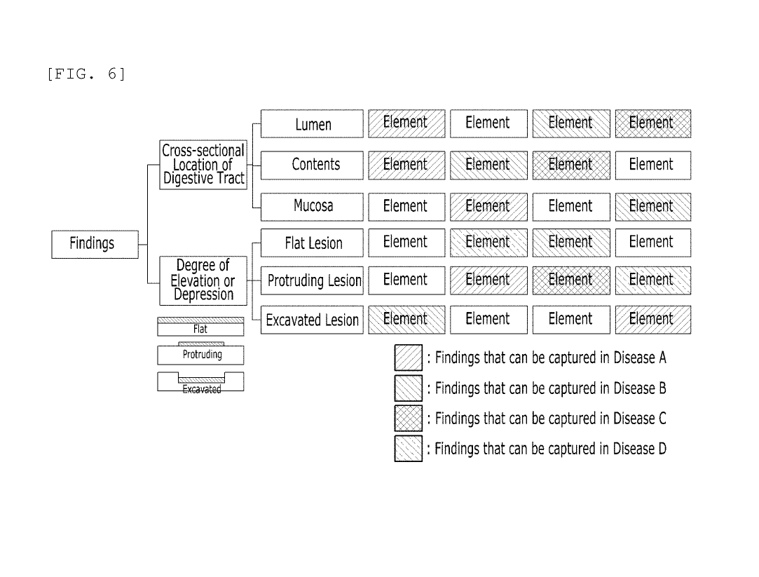

Next, referring to FIG. 5, it is understood that the relation between the signs and symptoms and the suspected diseases is defined by the knowledge model. The relation illustrated at the center of FIG. 5 illustrates the relation. Referring to FIG. 5, more specifically, normal test results of the patient and contents input through the medical interview are received from the medical information system such as EMR, PACS, or LIS to select a suspected disease. For example, when the patient has S01 of intestinal bleeding, D10 of angiectasia is selected as a suspected disease. As another example, when the patient has intestinal S01 of intestinal bleeding, S04 of anemia, and S06 of weight loss, D02 of celiac disease is selected as a suspected disease. As seen from the relation of FIG. 5, a suspected disease may be selected by a combination of a plurality of signs and symptoms.

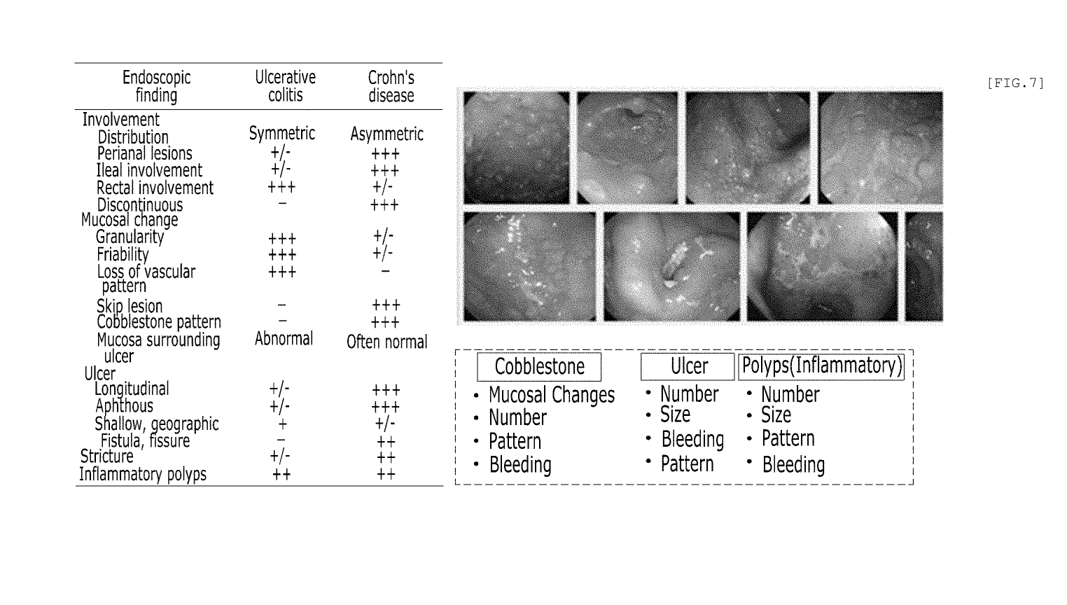

Next, referring to FIG. 6, it is understood that clinical findings to be found from the capsule endoscope proposed by MST are classified. Even though MST of the related art defines endoscopic findings as a simple list form, the knowledge model proposed by the present disclosure systemizes the findings and defines the relation between the disease and the findings based on the ontology. When the capsule endoscopy is read, finding information on the suspected diseases is considered. MST and CEST define finding information to be considered when the endoscopy is performed and the finding information has a structure as illustrated in FIG. 6. However, MST and CEST define the finding information without considering the relation for the disease. The meaning of findings in the present disclosure are the definitions of terminologies or attributes that can be used to write medical opinions or may be found from the endoscopic report. So that in the present disclosure, the relation of finding information on the disease is defined as illustrated in FIG. 7.

For example, in the case of Crohn's disease, as main opinion information, a longitudinal ulcer is observed or a cobblestone appearance or inflammatory polyposis, non-caseating epitheloid cell granuloma may be observed. Such information is illustrated in Table on a left side of FIG. 7. The relation between the signs and symptoms and the suspected diseases of FIG. 5 and the relation between the suspected disease and the endoscopic findings of FIG. 7 may be defined using the ontology.

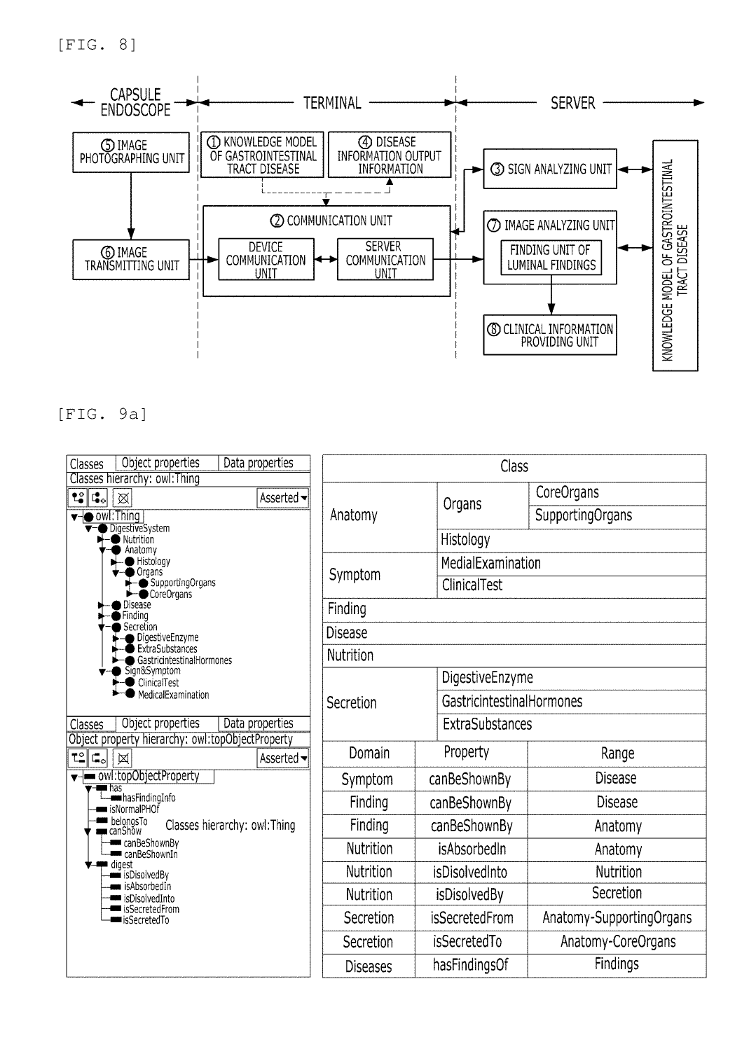

FIG. 8 is a view for explaining an image analyzing apparatus of a capsule endoscope based on a disease model according to an exemplary embodiment of the present disclosure.

An image analyzing apparatus of a capsule endoscope based on a disease model proposed by the present disclosure is referred to as a terminal in FIG. 8. The terminal may be a physical form such as a tablet, a smart phone, a wearable device, or a personal computer. The terminal receives images photographed by a capsule endoscope through an image transmitting unit of the capsule endoscope and short-range communication in real time. To this end, the terminal may include a communication unit and the communication unit of the terminal may include a device communication unit and a server communication unit.

For example, a short-range communication module such as NFC or Bluetooth of a smart phone is used as a device communication unit to receive the image photographed by the capsule endoscope. A long-range communication module such as 3G or LTE may exchange data with a server. Information exchanging between the terminal and the server is information for image analysis. That is, when the images received from the capsule endoscope are periodically or aperiodically transmitted from the terminal to server, the server detects endoscopic findings based on a knowledge model of gastrointestinal tract diseases by an image analyzing unit and matches the findings and the suspected disease to provide clinical information.

By doing this, the terminal receives the clinical information by the server communication unit to provide the clinical information to the patient or the medical staff through a disease information output interface. In addition, the terminal may receive sign information from the patient through a sign information input interface. In the example illustrated in FIG. 8, even though a centralized system in which a server analyzes the images based on the knowledge model and the terminal receives only the results is illustrated, a distributed system in which the terminal receives the knowledge model from the server to analyze the endoscopic images may also be implemented. The example of FIG. 8 is merely an example for more understanding of the present disclosure, but does not limit the present disclosure.

To be more specific, the server of FIG. 8 includes a sign analyzing unit, an image analyzing unit, and a clinical information providing unit. The sign analyzing unit receives and analyzes signs of the patient input from the terminal from the communication unit. The analyzed contents are reasoning of the suspected disease according to the sign and information on the reasoned suspected disease is transmitted to the terminal again. In this case, the analysis is performed based on the knowledge model of gastrointestinal tract diseases. The image analyzing unit receives images photographed by the capsule endoscope and other information from the terminal and analyzes the images and the information by the server. The analysis is a process of obtaining luminal findings available from the suspected disease and is performed based on the knowledge model of gastrointestinal tract diseases. The clinical information providing unit provides related clinical information to a doctor or an analyzing specialist based on the findings obtained by the image analyzing unit. The provided clinical information is attribute information on a findable lesion and includes a shape of an ideal lesion, the number of lesions, bleeding, and a size.

The knowledge model of gastrointestinal tract diseases built in a database of the server by ontology includes two types of data which are referred to as a first model and a second model. The first model is a model storing knowledge information on reasons for a capsule endoscope as disease information and defines a relation between the signs and symptoms and the disease which have been previously analyzed. The second model refers to a relation of the finding information analyzed based on MST and CEST with the suspected diseases.

When the image analyzing system of a capsule endoscope based on a knowledge model proposed by the present disclosure illustrated in FIG. 8 is used, clinical information necessary for diagnosis may be provided or recommended through a knowledge model which defines a relation on suspected diseases which is major reading information of the capsule endoscope. By doing this, intelligent software with convenience and economic feasibility may be developed by providing clinical information which assists the diagnosis to the existing software which just reproduces the capsule endoscopic images.

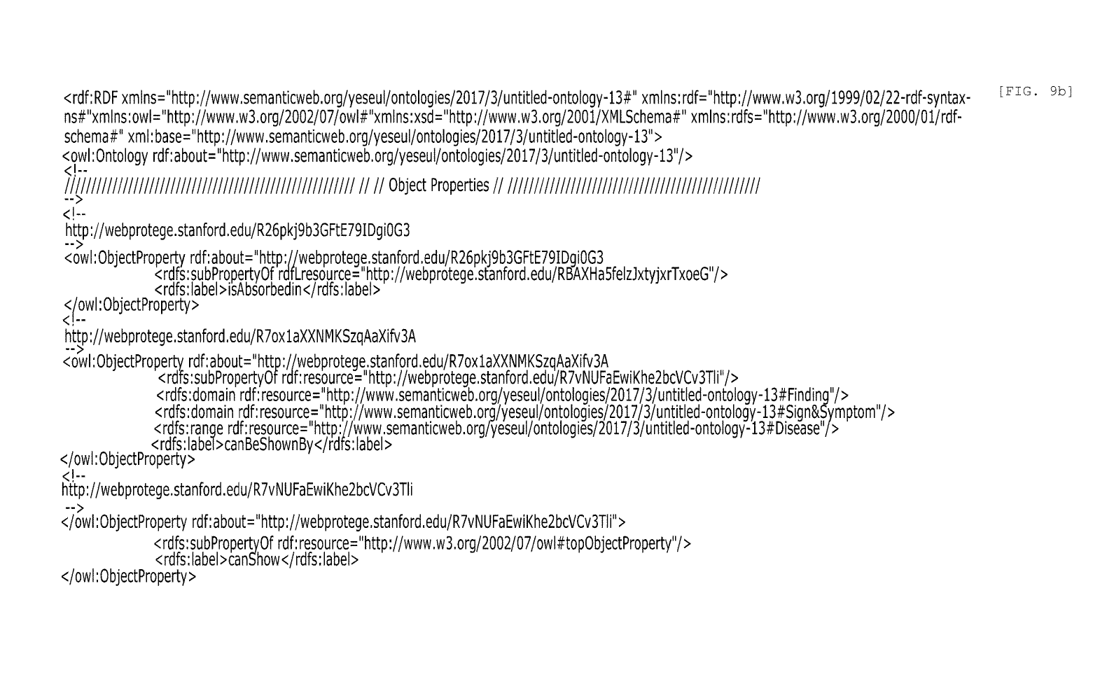

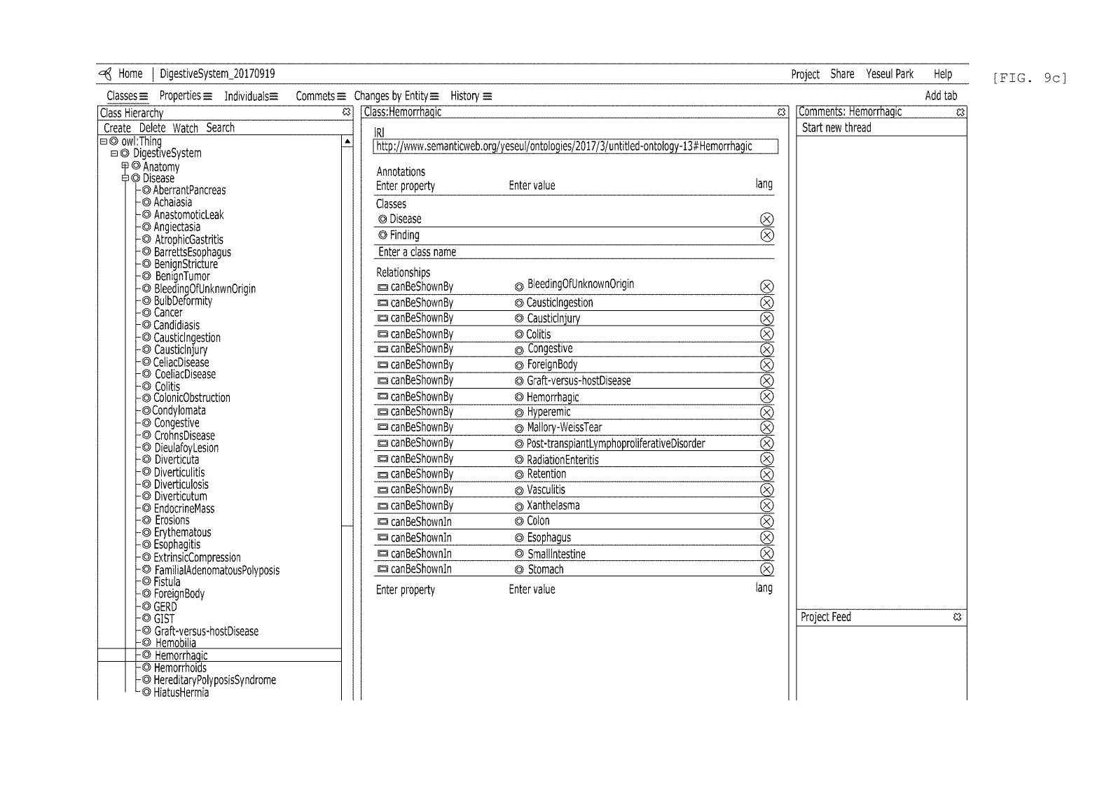

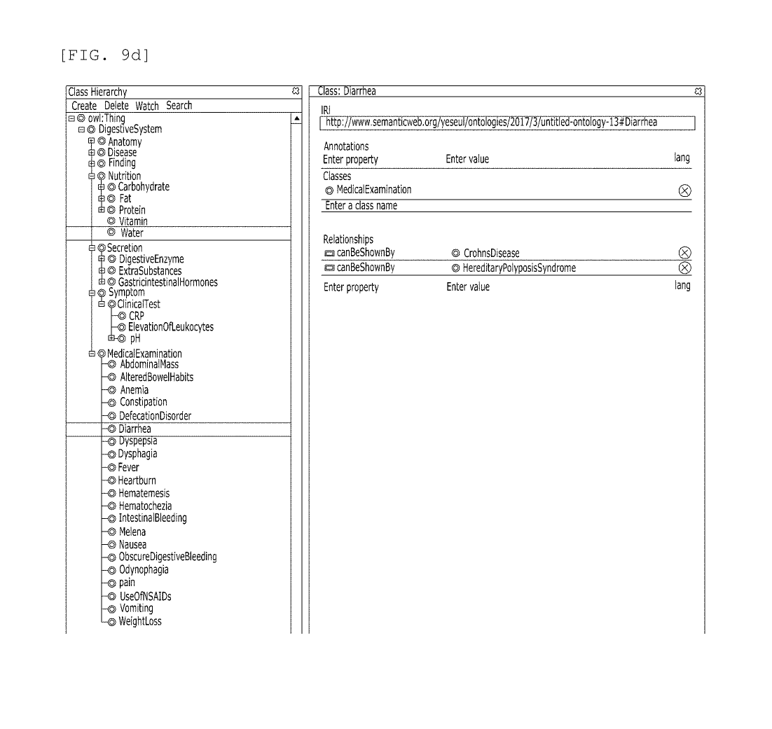

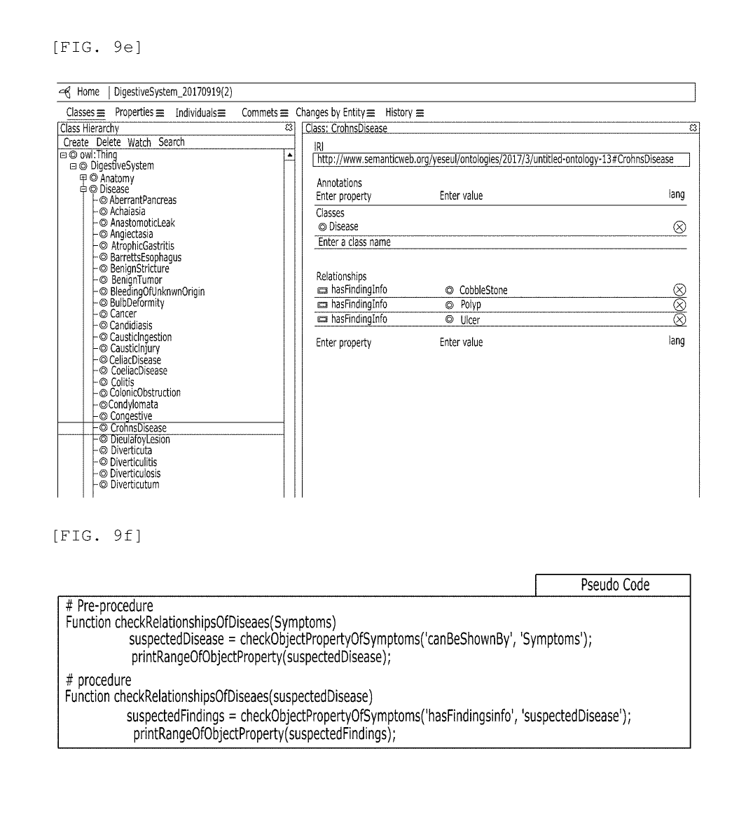

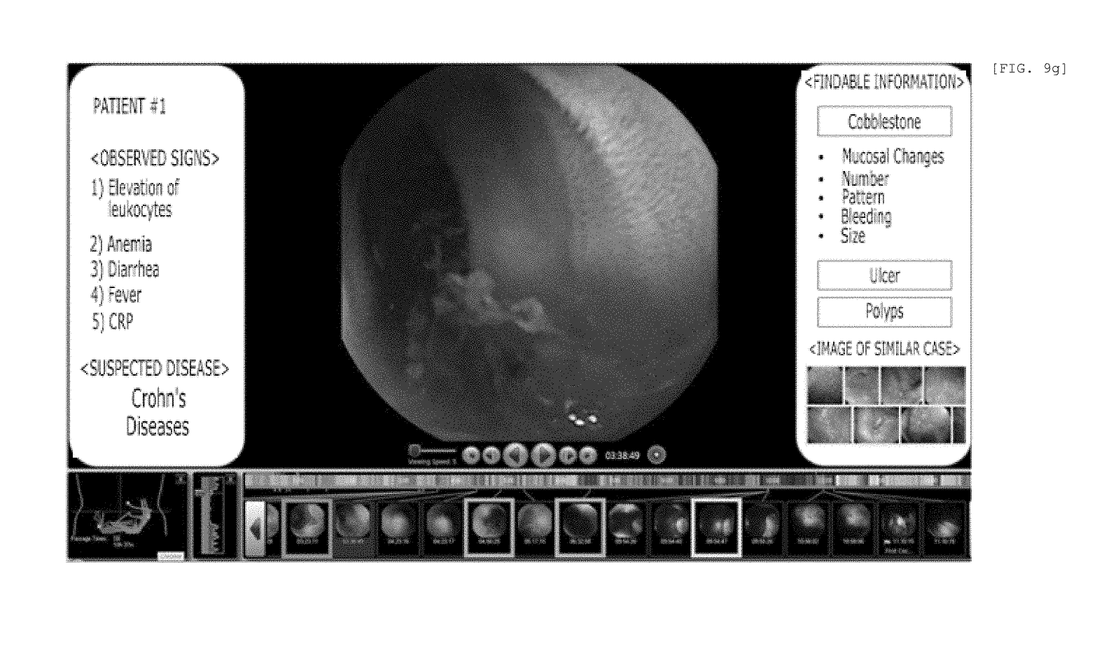

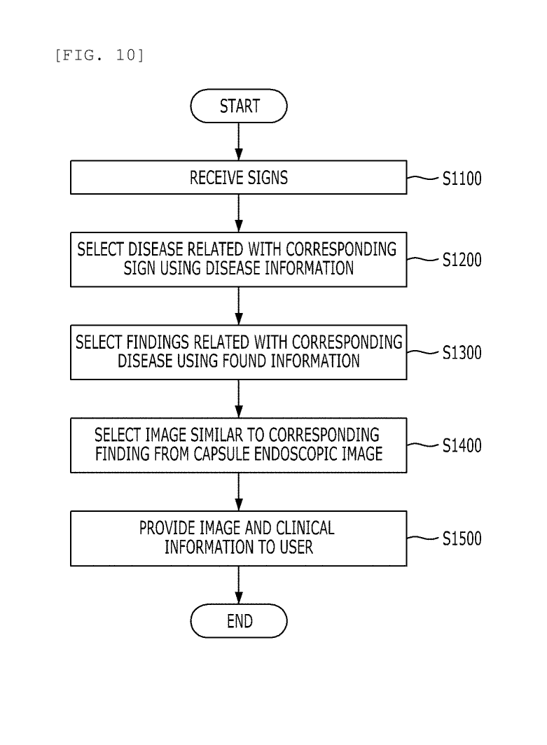

FIGS. 9A to 9G are views for explaining a knowledge model used in an exemplary embodiment of the present disclosure.

Referring to FIG. 9A, a knowledge model modeled by ontology may be identified. Here, the ontology may be defined as a relational model which expresses a relationship with other objects or a meaning of only the object to allow a computer to understand an arbitrary object (an object, a person, or whatever) like a person.

Specifically, in the domain of FIG. 9A, at a property of canBeShownBy (domain:Symptom, range:Disease), it may be defined that a specific symptom may be generated from a specific disease (canBeShownBy). Similarly, at a property of canBeShownBy(domain:Finding, range:Disease), it may be defined that a specific Finding may be generated from a specific disease (canBeShownBy). Further, at a property of canBeShownIn(domain:Finding, range:Anatomy), it may be defined that a specific Finding may be generated from a specific gastrointestinal tract (Anatomy) (canBeShownIn). In addition, nutrition and secretion information may also be built by ontology. Such an ontology modeling may be expressed by RDF/XML format as illustrated in FIG. 9B.

Referring to FIG. 9C, a specific example of the knowledge model built by the ontology may be identified. Referring to FIG. 9C, Disease domain may be seen from a tree-shaped data structure at the top. In the case of hemorrhagic among diseases, it is understood that hemorrhage may be caused by Colitis, Congestive, ForeignBody, Vasculitis, through the property of canBeShownBy. Further, it is also understood that the hemorrhage may occur in colon or stomach, through the property of canBeShownIn.

Next, referring to FIG. 9D, another specific example of the knowledge model built by the ontology may be identified. In the case of Diarrhea which is a sub category of MedicalExamination in a domain of Symptom, it is identified that Diarrhea may be caused by CrohnsDisease through the property of CanBeShownBy. Alternatively, it is identified that Diarrhea may also be caused by HereditaryPolyposisSyndrome.

Next, referring to FIG. 9E, still another specific example of the knowledge model built by the ontology may be identified. In the case of CrohnsDisease in the domain of Disease, it is identified that endoscopic findings found from CrohnsDisease include CobbleStone, Polyps, and Ulcer through the property of hasFindingInfo.

Next, referring to FIG. 9F, a pseudo code of an algorithm which analyzes images of a capsule endoscope through symptoms-suspected disease-endoscopic findings using a knowledge model built by the ontology is illustrated. Referring to FIG. 9F, a function of checkRelationshipsOfDiseases receives a symptom as a parameter to reason a suspected disease through a knowledge model. That is, the function is a function of inquiring disease information which is a first model. Next, the function of checkRelationshipsOfFindings receives a suspected disease as a parameter to reason findings seen from the images photographed by the capsule endoscope through a knowledge model.

It is possible to build an intelligent system which assists a diagnosis of a medical staff by providing capsule endoscopic frames of a patient who has finding information similar to finding information reasoned through a model among patients in various cases built in a database. As described above, when the images photographed by the capsule endoscope are automatically screened and provided, the medical staff may conveniently check without checking the entire images photographed by the capsule endoscope.

Referring to FIG. 9G, a user screen GUI of an image analysis software of a capsule endoscope implemented using the method proposed by the present disclosure is illustrated. Only a frame from which a specific finding is shown is selected from the entire photographed images and signs and suspected disease information thereby are also provided together with the images as seen from the left side. Further, the endoscopic findings seen from the capsule endoscopic images due to the suspected disease may also be provided at the right side. Therefore, the convenience of the medical staff may be enhanced by the intelligent analysis program.

FIG. 10 is a flowchart for explaining an image analyzing method of a capsule endoscope based on a disease model according to an exemplary embodiment of the present disclosure.

Referring to FIG. 10, signs are received from a patient who uses a capsule endoscope in step S1100. Further, the symptoms may be input by medical staffs or patient-related information and symptoms may be transmitted by interworking with the medical information system. Next, a disease related to the sign information input by the patient is selected using disease information which is a first model configuring a knowledge model in step S1200 and findings which may be found from patients with the corresponding disease are selected using finding information which is a second model in step S1400. Next, images in which the corresponding findings appear are selected in step S1400 and provided to the medical staffs through a GUI in step S1500.

The exemplary embodiments of the present disclosure have been described with reference to the accompanying drawings, but those skilled in the art will understand that the present disclosure may be implemented in another specific form without changing the technical spirit or an essential feature thereof. Thus, it is to be appreciated that the embodiments described above are intended to be illustrative in every sense, and not restrictive.

What is claimed is:

1. A capsule endoscopic image analyzing method, comprising:
receiving signs from a user, by a capsule endoscopic image analyzing apparatus;
determining a disease which shows the signs using disease information included in a knowledge model, by the capsule endoscopic image analyzing apparatus;
determining findings which are found from a gastrointestinal tract due to the disease using finding information included in the knowledge model, by the capsule endoscopic image analyzing apparatus; and
separately providing only frames in which the findings appear in images photographed by a capsule endoscope, by the capsule endoscopic image analyzing apparatus.

2. The capsule endoscopic image analyzing method according to claim 1, the method further comprising:
defining a relation between signs and a disease and a relation between the disease and findings in advance, based on the ontology, by the capsule endoscopic image analyzing apparatus.

3. The capsule endoscopic image analyzing method according to claim 1, wherein the separately providing of only frames includes: proposing clinical information to prepare a report, defined in capsule endoscopy structured terminology (CEST), including the frame as an image form and information on the disease and the findings as a text form.

4. The capsule endoscopic image analyzing method according to claim 3, wherein a plurality of findings is provided and the report further includes statistic information on the number and the size for the plurality of findings.

5. The capsule endoscopic image analyzing method according to claim 3, wherein a plurality of diseases is provided and the report further includes statistic information of an actual occurring probability of each disease according to a matching rate of each disease included in the plurality of diseases and the findings.

6. The capsule endoscopic image analyzing method according to claim 1, wherein the receiving of signs from a user includes: additionally, receiving symptoms from a medical information system.

7. The capsule endoscopic image analyzing method according to claim 1, wherein the separately providing of only frames includes: visually providing a first region which provides the overall image photographed by the capsule endoscope and a second region which separately displays only the frame around the first region through a graphic user interface (GUI).

8. A capsule endoscopic image analyzing apparatus, comprising:
an input unit which receives signs from a user;
a sign analyzing unit which determines a disease showing the signs using disease information included in a knowledge model;
an image analyzing unit which determines findings which are found from a gastrointestinal tract due to the disease using finding information included in the knowledge model; and
a disease information output unit which separately provides only frames in which the findings appear in an image photographed by a capsule endoscope.
